(19) United States
(12) Patent Application Publication
(10) Pub. No.: US 2022/0401418 A1
(43) Pub. Date: Dec. 22, 2022

(54) TREATMENT OF MALARIA USING HISTONE DEACETYLASE (HDAC) INHIBITORS

(71) Applicant: FOUNDATION FOR NEGLECTED DISEASE RESEARCH, Bangalore (IN)

(72) Inventors: Ganesh SAMBASIVAM, Bangalore (IN); Govinda Rajulu GAVARA, Bommasandra (IN); Vijay POTLURI, Hyderabad (IN); Rk SHANDIL, Bangalore (IN); Shridhar NARAYANAN, Bangalore (IN)

(21) Appl. No.: 17/774,813

(22) PCT Filed: Nov. 4, 2020
(86) PCT No.: PCT/IB2020/060351

§ 371 Date: May 5, 2022

(30) Foreign Application Priority Data
Nov. 7, 2019 (IN) 201941045323

(51) Int. Cl. A61K 31/4192; A61K 31/5377; A61P 33/06

(52) U.S. Cl. CPC A61K 31/4192; A61K 31/5377; A61P 33/06

(57) ABSTRACT

The present invention provides methods for treating malaria. The methods of the present invention comprise administering to a subject in need thereof a therapeutically effective amount of a pharmaceutical composition comprising aN-hydroxy-4-((4-(4-(pyrrolidin-1-ylmethyl)phenyl)-1H-1,2,3-triazol-1-yl)methyl)benzamide.

### CROSS-REFERENCE TO RELATED APPLICATIONS

This application is a National Stage application of the International Patent Application No. PCT/IB2020/060351, filed 4 Nov. 2020, titled TREATMENT OF MALARIA USING HISTONE DEACETYLASE (HDAC) INHIBITORS and published as WO 2021/090194, which claims priority to and the benefit of Indian Provisional Patent Application No. 201941045323 filed on 7 Nov. 2019, each of which is incorporated by reference herein in its entirety.

### FIELD OF INVENTION

The present invention is related to a method of treating malaria using a pharmaceutical composition comprising aN-hydroxy-4-((4-(4-(pyrrolidin-1-ylmethyl)phenyl)-1H-1,2,3-triazol-1-yl)methyl)benzamide.

### BACKGROUND OF THE INVENTION

Epigenetic mechanisms have been promising therapeutic targets for a variety of diseases ranging from cancer, cardiovascular diseases, inflammation and infection to name a few. The interaction between the histone acetyl transferases (HATs) and the histone deacetylases (HDACs) facilitate the structure of the intact DNA. The epigenetic mechanisms play a role in chromatin modifications, gene mutations in DNA, inactivation of DNA repair mechanisms, activation of oncogenes and also apoptosis.

Histone deacetylase family consists of 18 different isoforms HDAC (1-11) and SIRT (1-7). They are classified into Zinc dependent (Class 1, Class IIa, IIb) and NAD dependent (Class IV). Several drugs such as Vorinostat, Panabinostat, Belinostat, and Romidepsin have already been approved for the treatment of various types of cancers (Int J Mol Sci. 2017 July; 18: 1414).

The HDAC enzymes (known as lysine deacetylases KDACs) in parasites have been identified as an important target for treating drug resistant parasitic infections (PLoS Negl Trop Dis. 2015; 9:e0004026). A systematic study conducted by Wang et al, showed that subtle differences in the chemical structure can alter the functional activity of the molecule depending upon the different isoforms that a molecule acts on or the cellular distribution of the target enzyme (Chem Biol. 2015; 22:273-84).

Pfal HDAC family consists of at least 5 isoforms with the Pfal HDAC1 being identified as the major target of most antimalarial molecules (Mol Biochem Parasit. 2009; 164:9-25, J Med Chem. 2009; 52:2185-2187). More than a decade ago HDAC inhibitors were identified as a new class of compounds with a potential to target Plasmodium and other Apicomplexan parasites. Plasmodium falciparum: HDAC (PfHDAC) inhibition has been shown to inhibit asexual P. falciparum in erythrocytes (Antimicrob. Agents. Chemother., 2008, 52: 1454-61). There is evidence of HDAC inhibitors showing activity against multi drug resistant clinical isolates of Pf and Pv (Antimicrob. Agents. Chemother., 2011, 55:961-66). Treatment of P. falciparum parasites with HDAC inhibitors results in genome wide transcriptional alterations (Nat Biotechnol. 2010; 28:91-98, PLoS ONE. 2012; 7:e31847, PLoS pathogens. 2010; 6:e1000737) and altered PfHDAC1 expression has been found in P. falciparum parasite lines with reduced clinical susceptibility to artemisinin (BMC genomics. 2011; 12:391).

The structural diversity of HDAC inhibitors is limited to a few classes, such as cyclic peptides (Apicidin and its analogs), Hydroxamates (SAHA, TSA, WR301801) and benzamides (MS-275). Apicidin, a cyclic tetrapeptide was found to have an IC₅₀ of 200 nM in Pf but was not selective. However, replacing the indole in apicidin with quinolone increased the selectivity (up to ˜200 fold) for Pf in whole cell assay compared to activity obtained for mammalian cells. Hydroxamate based HDAC inhibitors showed more promising in vitro profiles. This class of inhibitors includes the class I/II HDAC inhibitors trichostatin A (TSA), suberoylanilidehydroxamic acid (SAHA, Vorinostat) and a sulfonylpyrrolehydroxamate (4SC-201, Resminostat), with Vorinostat and Resminostat being the HDAC inhibitors approved for cancer therapy. Most of these candidates exhibit either poor selectivity with respect to human HDACs and/or low bioavailability, these drawbacks prevent these candidates from being considered for anti-malarial therapy. Recently, several hydroxamic acid-based compounds with greater in vitro inhibitory potency against Pf parasites than Vorinostat and with varying improvements on selectivity were reported (J. Med. Chem, 2009, 51:3437-48; Antimicrob. Agents. Chemother., 2008, 52: 1454-61). These have been identified by screening compounds with variations to the basic structure of HDAC inhibitors. They comprise a small zinc binding group (ZBG) that accesses the active site zinc ion, a linker region capable of fitting the narrow, hydrophobic, tubular cavity leading from the ZBG to the HDAC surface, and a capping group that blocks the entrance to the active site cavity. Hydroxamate compounds based on an L-cysteine (thioether in the linker region) or 2-aminosubericacid (methylene group in the linker region) scaffold show similar in vitro anti-parasitic potency (IC₅₀<200 nM), however better selectivity for Pfversus mammalian cells was generally observed for the 2-aminosuberic acid (ASA) compounds. Three compounds from phenyl-thiazolyl-hydroxamate-based HDAC inhibitor class were highly potent (IC50<3 nM) with high selectivity indices of >600. WR301801, a lead compound from this panel had an IC₅₀ of 0.5-1.5 nM against several drug-resistant lines of Pf; hyperacetylated Pfhistones in situ, and inhibited deactylase activity in Pf nuclear extracts (Antimicrob Agents Chemother., 2008, 52, 3467-77). However, owing to poor pharmacokinetic properties, the existing HDAC inhibitors are administered intravenously. There is overwhelming evidence to support the use of HDAC inhibitors in anti-malarial therapy, if a few issues with respect to pharmacokinetics, bioavailability and selectivity are addressed. Pf HDAC1 inhibitors have shown activity against three life cycle stages of the parasite (asexual, exo-erythrocytic and gametocyte stages) (Eu. J. Med. Chem., 201482, 204-213).

Histone deacetylase (HDAC) inhibitors are disclosed in US20120101099 which is incorporated herein by reference in its entirety. The US application provides compositions of HDAC inhibitors and their utility in cancer therapy. The present invention provides the use of HDAC inhibitors in the treatment of malaria and malarial infections.

### BRIEF DESCRIPTION OF DRAWINGS

FIG. 1: FNDR-20123 Parasite Killing Profile

FIGS. 2A and 2B: (2a) Oral treatment period; (2b) Reduction in parasitemia in humanized SCID mouse model of P. falciparum malaria

FIGS. 3A and 3B: (3a) Therapeutic efficacy in SCID mouse model of human P. falciparum malaria; (3b) Percentage reduction in parasitemia with respect to (wrt) infected control.

### DETAILED DESCRIPTION OF THE INVENTION

The present invention provides a compound of Formula (I)

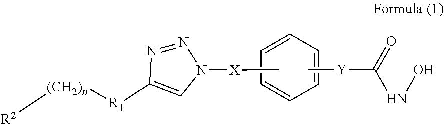

its derivatives, analogs, tautomeric forms, stereoisomers; polymorphs, solvates, salts, metabolites and prodrugs wherein,

R¹ is selected from a group comprising hydrogen, alkyl, alkenyl, alkynyl, haloalkyl, haloalkenyl, haloalkynyl, cycloalkyl, cycloalkenyl, heterocycloalkyl, heterocy cloalkenyl, heterocycloalkynyl, cycloalkylalkyl, hetero cycloalkylalkyl, arylalkyl, heteroarylalkyl, arylalkenyl, heteroarylalkenyl, arylalkynyl, heteroaryl alkynyl, cycloalkylheteroalky 1, arylheteroalkyl, heteroarylhet eroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl and heteroaryl;

(CH₂)ₙ, wherein n=0, 1, 2, 3; and

R² is selected from a group comprising hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl and heteroarylcarbonyl; and its use in the treatment of Malarial infections.

More specifically, compounds of Formula (I) include:

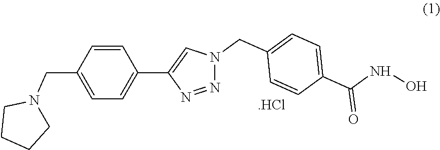 [SMILES: O=C(NO)c1ccc(Cn2cc(-c3ccc(CN4CCCC4)cc3)nn2)cc1 ; ".HCl"]

Compound (1) in this disclosure is referred to as FNDR-20123 or 20123.

The invention further includes Compounds (2) to (6) as provided below:

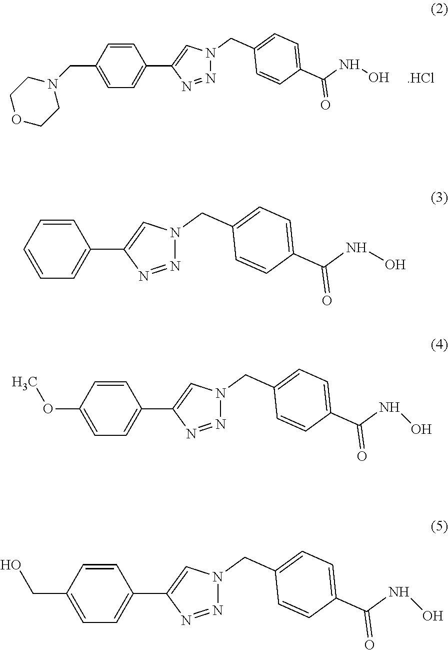 [SMILES: O=C(NO)c1ccc(Cn2cc(-c3ccc(CN4CCOCC4)cc3)nn2)cc1 ; ".HCl" ; O=C(NO)c1ccc(Cn2cc(-c3ccccc3)nn2)cc1 ; COc1ccc(-c2cn(Cc3ccc(C(=O)NO)cc3)nn2)cc1 ; O=C(NO)c1ccc(Cn2cc(-c3ccc(CO)cc3)nn2)cc1]

The invention particularly provides use of HDAC inhibitors, as provided in Formula (I) and Compounds (1) to (5) in the treatment of infections caused by Malarial parasite. Herein, the infection includes all kinds of malarial infections such as blood, liver-stage and cerebral malaria showing symptoms of fever, chills, headache, nausea, vomiting, muscle pain, fatigue, sweating, chest or abdominal pain and cough caused by the malarial parasite species of P. falciparum, P. malariae, P. vivax, P. ovale and P. knowlesi.

The invention also pertains to the use of a compound of Formula (I) and Compounds (1) to (5), its derivatives, analogs, tautomeric forms, stereoisomers; polymorphs, solvates, salts, metabolites and prodrugs, in the manufacture of a medicament for the treatment of infections caused by malarial parasite.

The invention also provides the use of a compound of Formula (I) and Compounds (1) to (5), or a pharmaceutically acceptable salt thereof, in the manufacture of a medicament for the treatment of infections caused by malarial parasite.

In one aspect, the invention pertains to the use of a compound of Formula (I) and Compounds (1) to (5), or a pharmaceutically acceptable salt thereof, and a pharmaceutically acceptable excipient, diluents or carrier.

Uses

The compounds of the invention are useful for the treatment of malarial infections in subjects, mammals in particular, including humans. In one embodiment, the compounds may be used for the treatment of infections of brain, blood and liver.

In another embodiment, the compounds of the invention are useful for the treatment of human infections including but not limited to, lung infections, endocarditis, blood stream infections, surgical site infections and infections associated with intravascular devices caused by microorganisms, such as but not limited to, Plasmodium sps including P. falciparum, P. malariae, P. vivax, P. ovale and P. knowlesi.

Route of Administration

The compounds of the present invention are delivered to the subjects by forms suitable for each administration route. For example, the compounds are administered as tablets, capsules, injection, drops, inhaler, ointment, foams suppository. In a preferred embodiment, the route of administration is oral, parenteral or topical. Topical or transdermal administration includes powders, sprays, ointments, pastes creams, lotions, gels, solutions, patches and inhalants. Most preferably the route of administration is parenteral route.

Dosage Forms for Parenteral & Oral Routes

The compositions of the present invention are presented in unit dosage forms generally in an amount that produces a therapeutic effect in the subject.

The compounds of the present invention are administered at a daily dose that is the lowest dose effective to produce a therapeutic effect. Generally, the dosage will effect from about 0.0001 to about 100 mg per kg body weight per day. Preferably, the dosage will range from about 0.001 to 75 mg per kg body weight per day and more preferably, the dosage will range from about 0.1 to about 50 mg per kg body weight per day. Each unit dose may be, for example, 5, 10, 25, 50, 100, 125, 150, 200 or 250 mg of the compound of the invention. As per the requirement of the subject, the effective daily dose of the compound is administered as two, three, four or more sub-doses administered separately at appropriate intervals throughout the day, optionally in unit dosage forms.

Formulation

The antimalarial compositions of the present invention may be administered by any method known in the art. Some examples of suitable modes of administration include oral, intravenous, intramuscular topical or any other parenteral mode of administration.

In certain embodiments, the present invention is directed to a method of formulating compounds of the present invention in a pharmaceutically acceptable carrier or excipient and may be administered in a wide variety of different dosage forms e.g. tablets, capsules, sprays, creams, lotions, ointments, aqueous suspensions syrups, and the like. Such carriers may include one or more of solid diluents or fillers, sterile aqueous media, and various nontoxic organic solvents, etc.

For oral administration, tablets may contain various excipients such as one or more of microcrystalline cellulose, sodium citrate, calcium carbonate and the like, along with various disintegrants such as starch and certain complex silicates, together with granulation binders like polyvinylpyrrolidone, sucrose and the like. Solid compositions of a similar type may also be employed as fillers in gelatin capsules.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluents or solvent e.g. as solution in 1, 3 butane diol. Among the acceptable vehicles and solvents that may be employed are water, Ringer's solution and isotonic sodium chloride solution. In addition, sterile fixed oils are conventionally employed including synthetic mono or diglycerides. In addition, fatty acids such as oleic acid find in the preparation of injectables. These aqueous solutions may be suitable for intravenous injection purposes. The oily solutions may be suitable for intra articular, intramuscular, and/or subcutaneous injection purposes.

In another embodiment, the compounds of the present invention may be administered topically that include transdermal, buccal, or sublingual application. For topical applications, therapeutic compounds may be suitably admixed in a pharmacologically inert topical carrier such as a gel, an ointment, a lotion, and/or a cream. Such topical carriers may include water, glycerol, alcohol, propylene glycol, fatty alcohols, triglycerides, fatty acid esters, and/or mineral oils.

The timing of the administration of the pharmaceutical composition may also be regulated. For example the compounds may be administered intermittently or by controlled release.

### Definitions

As used herein, the term ‘alkyl’ refers to the radical of saturated aliphatic groups, including straight-chain alkyl groups, branched-chain alkyl groups, cycloalkyl (alicyclic) groups, alkyl substituted cycloalkyl groups and cycloalkyl substituted alkyl groups.

The compounds of present invention may exist in specific geometric or stereoisomeric forms.

The present invention is inclusive of all possible enantiomers and diastereomers in pure or substantially pure form and mixtures of two or more stereoisomers in ratios that are effective. This means that the compounds of present invention may exist both as levorotatory and as dextrorotatory, in the form of racemates and in the form of two enantiomers.

The compounds of present invention are capable of forming both pharmaceutically acceptable salts. Examples of salts include but not restricted to metals or amines such as alkali and alkaline earth metals or organic amines. Examples of suitable acids for salt formation include but not restricted to hydrochloric, sulphuric, phosphoric, acetic, citric, oxalic, malonic, salicyclic, malic, fumaric, succinic, ascorbic and the likes thereof.

The compound of the invention can exist as unsolvated or solvated forms including hydrated forms.

The compounds detailed in the present disclosure are capable of forming pharmaceutically acceptable prodrugs. Prodrugs are covalently bonded carriers that release the active compound in pharmaceutically acceptable form internally after administration to the subject.

The present invention provides pharmaceutical compositions comprising an effective amount of compound of Formula I prodrugs, tautomeric forms, stereoisomers, optical isomers, pharmaceutically acceptable salts, solvates or polymorphs thereof with pharmaceutically acceptable carriers.

The invention can be fully understood by reference to the following Examples. These examples, however, are not to be construed as limiting the scope of the invention.

### Examples

Methods

Malarial Parasite Culture Start-Up and Maintenance

Plasmodium falciparum 3D7 strain was used for the P. falciparum asexual blood-stage (ABS) assay, for which the cells were resuscitated from the stabilate and maintained at 5% haematocrit. Red blood cells obtained from the hospital were used to maintain the culture at 1% parasitaemia while screening.

### Example 1. Antimalarial Activity

Applicant's proprietary compound library was screened for anti-malarial activity and 10 actives (IC₅₀<500 nM) were identified of which FNDR-20123 showed potent anti-malarial activity.

HDAC Activity Screening

HDAC inhibition screening was performed using a fluorescence-based assay with a fluorescent substrate (BocLys (Ac)-AMC Substrate). HeLa nuclear extract was used as the enzyme source. For selected compounds, IC50 (50% HDAC inhibitory concentration) was determined by testing in a broad concentration range of 0.001, 0.01, 0.1, 1 and 10 μM.

The assay was performed in 96-well black microplates, and the total volume of the assay was set at 100 μl. Briefly, HeLa nuclear extract was diluted with HDAC assay buffer (final concentration of 30 μM), containing 25 mM Tris/Cl, pH 8.0, 137 mM NaCl, 2.7 mM KCl, and 1 mM MgCl2. The enzyme mixture was prepared by adding 10 mM of the diluted enzyme (HeLa nuclear extract) to 30 μM of HDAC buffer. From the enzyme mixture, 40 μl was taken and mixed with 10 μl of test compound (final concentration from 0.01 to 10 μM) or vehicle (control). The final mixture (50 μl) was added to each well which was then pre-incubated at 37° C. for 10 min. The HDAC reaction was started by adding 50 μl of HDAC substrate: Boc-Lys (Ac)-AMC (Anaspec, Inc Fremont, Calif., USA). The plate was incubated at 37° C. for 45 min. Trypsin stop solution (50 μl) was added to the well, and the plate was further incubated at 37° C. for 15 min to stop the reaction. The release of AMC was monitored by measuring the fluorescence at an excitation wavelength of 360 nm and an emission wavelength of 460 nm. Buffer and substrate alone served as blank.

Isoform selectivity was tested using recombinant HDAC isoforms (Biomol, USA). FNDR-20123 was tested against human HDAC1, HDAC2, HDAC3, HDAC6, and HDAC8 isoforms.

The activity of FNDR-20123 against PfHDAC was assessed with a HDAC fluorescent activity assay kit (BPS-Bioscience's pf-HDAC1, Malaria, His-tag, FLAG-tag FNDR-20123 was dissolved in 100% DMSO and stored in −20° C. until use. Enzyme concentration (PfHDAC1) was optimized to 4 ng/μL to get detectable activity. TSA was used as a control in the assay to determine the inhibitory activity. Briefly, HDAC assay buffer (25 mM Tris/Cl, pH 8.0, 137 mM NaCl, 2.7 mM KCl, and 1 mM MgCl2), BSA, HDAC substrate and PfHDAC1 enzyme were mixed in an amber colour 96-well plate and incubated at 37° C. for 30 min. After incubation, the reaction was stopped by adding 50 μl of HDAC assay developer and the plate was incubated further at room temperature for 30 min. The fluorescence developed was measured with excitation at 350-380 nm and emission at 440-460 nm.

Results of this assay are provided in Table-1.

Plasmodium falciparum: Asexual Blood-Stage (ABS) Assay

Plasmodium falciparum: 3D7 cells were used as a target strain for the assay. Mefloquine (Sigma-Aldrich) was used as a standard inhibitor. At day 1, 250 ml of 10 mM mefloquine was added to columns 12 and 24 of the sterile 384-well black, clear-bottom, cell culture assay plates followed by compound curves which were added using Echo. The P. falciparum 3D7 cells were counted and 20 ml of culture was prepared at 5% haematocrit, 0.3% parasitaemia (as one batch, i.e., 12 plates, prepare 260 ml, to allow for WellMate prime volume) for each plate. From this, 50 μl of culture was added to all wells on all plates using WellMate with small bore tubing on full (S-1) speed. The plates were placed on the bottom shelf of the incubator, with a maximum stack height of 4 plates, preferring the front of the shelf and were incubated for 72 h at 37° C. in an atmosphere of special gas mix (1% O₂, 3% CO₂, balance N2).

On day 4, SybrGreen/lysis buffer was prepared by diluting defrosted SybrGreen aliquots as required (20 μl of 10,000×req. for 12 plates) to 3× with lysis buffer (70 ml for 12 plates). Ten μl of the prepared buffer was added to each well on each of the assay plates and incubated overnight in the dark at room temperature. The plates were read on Victor plate reader using ‘384sybrgreen’ protocol (excitation 485 nm, emission 528 nm) after which the plate contents were aspirated into 5% Virkon, and disposed of after autoclaving. Percentage inhibition for each test compound was calculated using the following equation:

% Inhibition=100−(TEST COMPOUND−BLANK)/(NO INHIBITION−BLANK)*100)

Results of this assay are provided in Table-1.

Male and Female Gametocyte Functional Viability Assay

FNDR-20123 was assessed in male and female gametocyte functional viability assay. Briefly, gametocyte cultures were seeded at 1% rings and 4% haematocrit under 3% O₂, 5% CO₂, 92% N₂ gas by using an asexual culture with 3% ring stages at day 0. Gametocyte cultures were tested for functional viability and maturity after 14 days. Testing functional viability was done by quantifying male gametocyte formation, which was carried out by withdrawing 200 μl of culture. Following this, culture was centrifuged, and the pellet was resuspended in 5 μl ookinete medium (RPMI medium with 25 mM HEPES, 50 mg/l hypoxanthine, 2 g/l sodium bicarbonate, 100 μM xanthurenic acid, and 10% A+human serum). The culture was observed under a microscope. After validating maturity and upon exflagellation centres being >50 per field, the gametocyte culture was resuspended in 7.5 ml complete medium. From this, 50 μl was dispersed into previously prepared 96-well plate (containing complete culture medium and FNDR20123 at a concentration of 1 μM and prewarmed at 37° C. for 20 min). The plates were then incubated at 37° C. for 24 h. Gamete formation was induced on day 15 and observed under the microscope. The assay was performed using four independent biological replicates.

Results of this assay are provided in Table-1.

The results show that FNDR-20123 inhibits Pf-HDAC1 and Human HDAC with IC₅₀ of about 30 and 3 nM respectively. While the antimalarial activity in asexual blood stage was found to be 41 nM, it exhibited gametocicdal activity against male gametocytes with an IC₅₀ of 190 nM (Table-1).

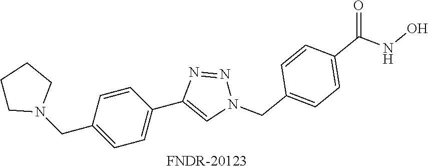

TABLE 1

| | | | | Hu HDAC | | | |
|---|---|---|---|---|---|---|---|
| | Physicochemical | | | IC₅₀ (nM) | Pf- | Anti-Malarial Activity | |
| | Properties | | | Hela | HDAC1 | Asexual Blood | Gametocytes IC₅₀ |
| | Mol. | | | Nuclear | IC₅₀ | Stage | (nM) |
| Compound | Wt. | ClogP | PSA | Extract | (nM) | IC₅₀ (nM) | Male/Female |
| 20123 | 377.45 | 1.917 | 80.53 | 3 | 31 | 41 | 190/>5000 |

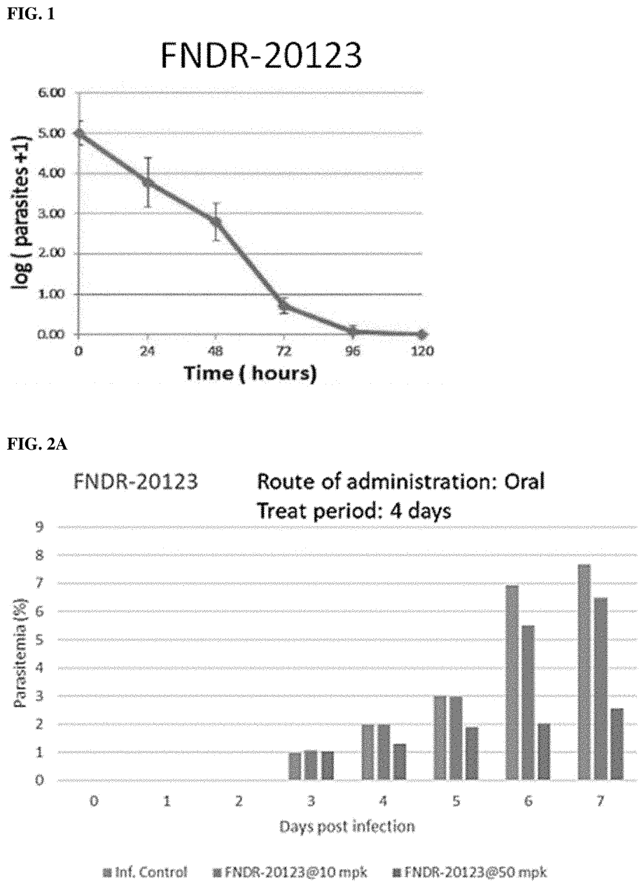

FIG. 1 shows the in vitro killing profile of FNDR-20123. FNDR-20123 is found to be potent against all the mutant/strains of P. falciparum tested (Tables 2a & 2b).

FNDR-20123 also shows good liver microsomal stability, low plasma protein binding across species, no hERG liability and no inhibition of the CYP isoforms tested (Table-3).

TABLE 2a

| | | | FNDR-20123 | |
|---|---|---|---|---|
| | | | | Fold shift |
| | Mutated | | IC₅₀ | IC₅₀ rel. |
| | locus | Mutations (amino acid changes) | (nM) | Dd2 |
| Dd2 | Wild | N/A | 17.50 | 1.0 |
| | type | | | |
| Dd2 | PfeEF2 | Y186N (Baragaña et al. 2015 Nature) | 20.86 | 1.2 |
| DDD107498 | | | | |
| Dd2 | Pfpi4k | S743T (Paquet et al., 2017 Sci Transl Med) | 18.27 | 1.0 |
| 390048 | | | | |
| Dd2 | Pfdhodh | G181C | 17.83 | 1.0 |
| DSM265 | | (Philipps et al 2015 Sci Transl Med) | | |
| Dd2 | Pfcarl | Ile1139Lys (Meister et al. 2011 Science) | 18.45 | 1.1 |
| GNF156 | | | | |
| Dd2 | PfcytB | Ile22Leu (Stickles et al. 2015 AAC) | 13.49 | 0.8 |
| ELQ300 | | | | |

TABLE 2b

FNDR-20123

| | IC₅₀ (nM) | Fold shift IC₅₀ rel. NF54 |
|---|---|---|
| NF54 | 25.27 | 1.0 |
| K1 | 19.05 | 0.75 |
| 7G8 | 14.36 | 0.57 |
| TM90C2B | 13.26 | 0.52 |
| Cam3.I (MRA1240) | 22.06 | 0.87 |
| Dd2 | 17.50 | 0.69 |

Method Reference for Table 2a and 2b: Marfurt J, et al. Ex vivo activity of histone deacetylase inhibitors against multidrug-resistant clinical isolates of Plasmodium falciparum and P. vivax. Antimicrob Agents Chemother. 2011; 55(3):961-966.

TABLE 3

| | | FNDR-20123 |
|---|---|---|
| Plasma Protein Binding (% | Mouse/Rat/Dog/Monkey/ | <30.00/41.89/<20.00/<20.00/57.30 |
| Bound) | Human | |
| Microsomal Stability | MLM/RLM/HLM | 76.15/74.56/85.31 |
| (% remaining at 120 min) | (Test Conc. 10 μM) | |
| Plasma Stability | Mouse/Rat/Dog/Human | 89.16/88.90/97.72/98.94 |
| | (% remaining at 240 | |
| | min) | |
| Cyp Inhibition (IC₅₀ μM) | CYP 1A2 | >60 |
| | CYP 2C9 | >60 |
| | CYP 2C19 | >60 |
| | CYP 2D6 | 28.00 |
| | CYP 3A4 | 27.00 |
| CYP induction (% induction | CYP 1A2 | 2.8 ± 0.8 |
| relative to respective positive | CYP 2B6 | 0.0 |
| controls) | CYP 3A4 | 0.9 ± 1.5 |
| Cytotoxicity | HepG2 (% @ 100 μM) | 12.6% |
| | THP-1 (IC₅₀ μM) | 113.6 μM |
| HERG Binding | IC₅₀ in μM | >100 μM |

Method Reference for Table 3: Chung T D Y et al. In Vitro and In Vivo Assessment of ADME and PK Properties during lead selection and lead optimisation-guidelines, benchmarks and rules of thumb. In: Assay Guidance Manual. 2015.

### Example 2. In Vivo Efficacy

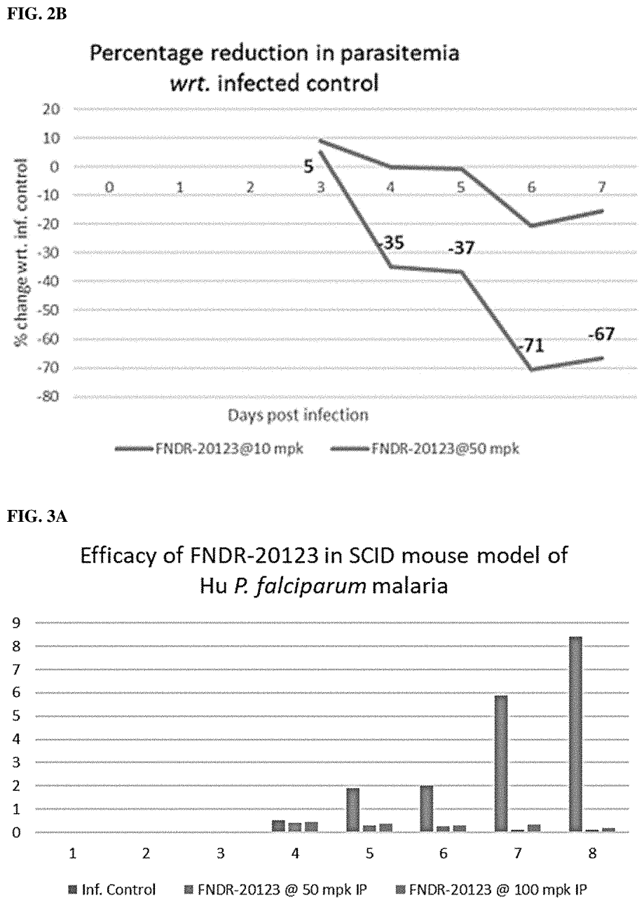

In vivo efficacy assessed in humanized SCID mouse model of P. falciparum malaria. FNDR-20123 was dosed orally at 10 and 50 mg/kg body weight for 4 days. FNDR-20123 showed 67% reduction in parasitemia at 50 mg/kg body weight (FIGS. 2A and 2B).

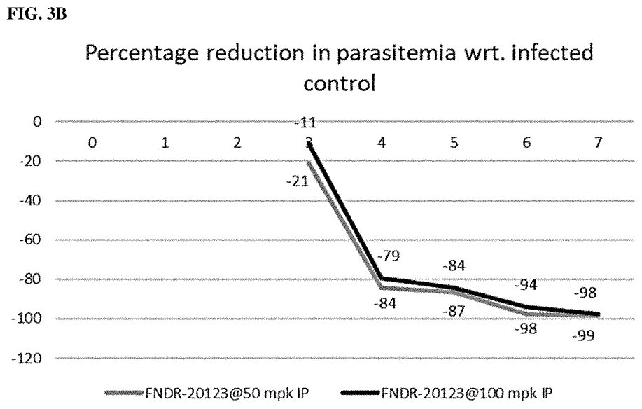

FNDR-20123 was administered intra-peritoneally at 50 and 100 mg/kg to humanized SCID mice infected with P. falciparum malaria. 98% and 99% reduction in parasitaemia was seen at 50 and 100 mg/kg doses respectively (FIGS. 3A and 3B).

In conclusion, the present invention provides a safe and potent Pf HDAC inhibitor with good antimalarial activity as seen in in vitro and in vivo studies.

1. A method of treating malarial infections in a subject, the method comprising administering to the subject in need thereof an effective amount of a compound of Formula (I): 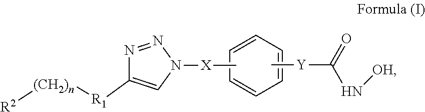
or a derivative, analog, tautomeric form, stereoisomer, polymorph, solvate, pharmaceutically acceptable salt, metabolite, or prodrug thereof,
wherein,
the compound is effective in treating the malarial infection;
R¹ is selected from hydrogen, alkyl, alkenyl, alkynyl, haloalkyl, haloalkenyl, haloalkynyl, cycloalkyl, cycloalkenyl, heterocycloalkyl, heterocycloalkenyl, heterocycloalkynyl, cycloalkylalkyl, heterocycloalkylalkyl, arylalkyl, heteroarylalkyl, arylalkenyl, heteroarylalkenyl, arylalkynyl, heteroaryl alkynyl, cycloalkylheteroalkyl, arylheteroalkyl, heteroarylheteroalkyl, heterocycloalkylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, COOH, alkoxycarbonyl, alkylaminocarbonyl, arylaminocarbonyl, heteroarylcarbonyl, aryl, and heteroaryl;
n is 0, 1, 2, or 3; and
R is selected from hydrogen, halogen, alkyl, cycloalkyl, heterocyclyl, aryl, aralkyl, heteroaryl, amino, alkylamino, aminoalkyl, alkylaminoalkyl, acylamino, arylamino, alkoxycarbonyl, alkylaminocarbonyl, arylaminocarbonyl, and heteroarylcarbonyl.

2. The method of claim 1, wherein the compound is selected from:

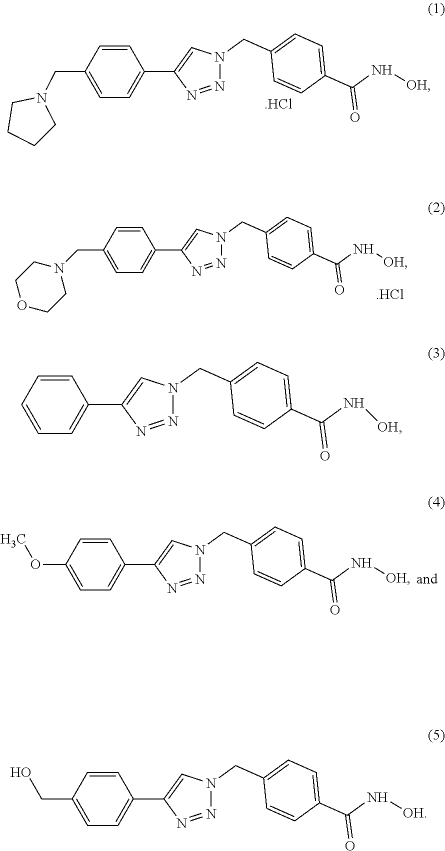

3. The method of claim 1, wherein the malarial infection is caused by Plasmodium falciparum, Plasmodium malariae, Plasmodium vivax, Plasmodium ovale, or Plasmodium knowlesi.

4. The method of claim 2, wherein the malarial infection is caused by Plasmodium falciparum, Plasmodium malariae, Plasmodium vivax, Plasmodium ovale, or Plasmodium knowlesi.

5. A composition comprising the compound of claim 1 or a pharmaceutically acceptable salt thereof, and a pharmaceutically acceptable excipient, diluent, or carrier.

6. The composition of claim 5, wherein the compound is selected from:

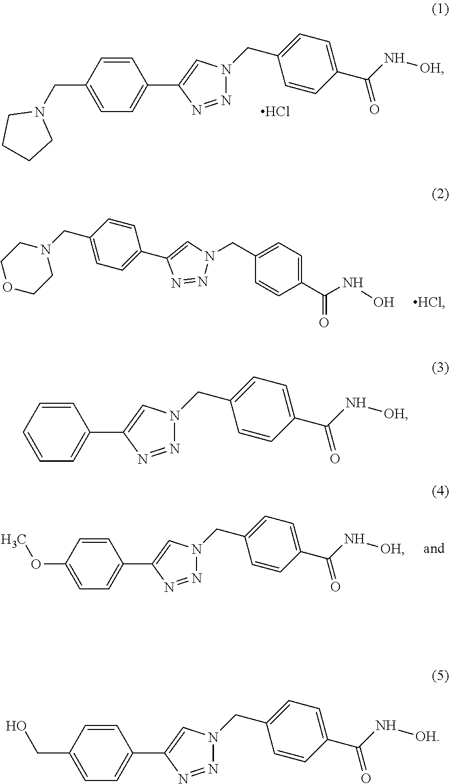

7. The method of claim 1, wherein the malarial invention is caused by Plasmodium falciparum.

8. The method of claim 2, wherein the malarial invention is caused by Plasmodium falciparum.
